(19) United States
(12) Patent Application Publication
(10) Pub. No.: US 2012/0323319 A1
(43) Pub. Date: Dec. 20, 2012

(54) INTRACORNEAL DIFFRACTIVE LENS HAVING PHASE INVERSION

(76) Inventors: Gilbert Cohen, Lyon (FR); Fannie Castignoles, Villeurbanne (FR); Thierry Lepine, Saint Heand (FR)

(21) Appl. No.: 13/508,157

(22) PCT Filed: Oct. 29, 2010
(86) PCT No.: PCT/FR2010/052323

§ 371 Date: Aug. 15, 2012

(30) Foreign Application Priority Data
Nov. 6, 2009 (FR) 0957865

(51) Int. Cl. A61F 2/14

(52) U.S. Cl. 623/5.11

(57) ABSTRACT

The invention relates to an intracorneal diffractive lens having phase inversion, said intracorneal diffractive lens including: a core (2) having a first surface and a second surface opposite the first surface; at least one first hydrogel layer (5) extending over the first surface of the core; and a second hydrogel layer (6) extending over the second surface of the core. The first hydrogel layer (5) includes, on the surface thereof turned toward the core, a plurality of concentrically or coaxially projecting annular areas (7), each annular area (7) having a continuously varying thickness toward the periphery of the lens. The first and second layers (5, 6) have a nutrient and oxygen permeability substantially identical to that of the corneal tissue, and at least one of the annular areas (7) of the first layer (5) is in contact with the second hydrogel layer (6).

### TECHNICAL FIELD

The present invention relates to intracorneal diffractive lenses that are intended to be placed in the cornea to correct vision defects, also called ametropias. More particularly, this invention relates to an intracorneal diffractive lens that can be used for the surgical correction of presbyopia.

### BRIEF DESCRIPTION OF RELATED ART

In the field of ametropia correction using refractive surgery, a distinction is made between corneal refractive surgery and endocular surgery, corneal surgery having fewer complications.

Currently, corneal refractive surgery is done by modifying the curvature of the front surface of the cornea.

More particularly, presbytia correction using corneal surgery is based on pseudo-accommodation, i.e. on the transformation of the cornea into a multifocal diopter by modifying the curvature of the cornea; in this refractive correction mode, the optic performance depends on the pupil diameter and the centering of the lens, and therefore the illumination level. In presbytia correction using endocular surgery, the use of diffractive lenses yields good results, independent of the centering of the lens and the pupil diameter.

Transforming the cornea into a diffractive lens by sculpture is not possible. Depending on the use thereof, an intracorneal diffractive lens would make it possible to benefit from the optic properties of diffractive lenses and the harmlessness of corneal surgery.

The current obstacles to the use of intracorneal implants, in particular intracorneal diffractive lenses, particularly to treat presbytia, are the biocompatibility of these implants and above all their permeability to the flows of nutrients and oxygen in the thickness of the cornea, this permeability being crucial to maintaining the transparency and refractive function of the cornea.

Documents EP 0420549 A2 and WO 99/07309 show examples of intracorneal diffractive lenses with phase inversion, made from hydrogels and comprising concentric annular areas, arranged in steps.

It is known to use hydrogels with a high water content (with a low optic index). Such hydrogels have good nutrient and oxygen permeability, but, however, have a low mechanical strength, which harms the stability of the architecture of the lens and the manipulation thereof.

It is also known to use hydrogels with a low water content (with a high optic index). Such hydrogels have good mechanical strength, but nevertheless have low nutrient and oxygen permeability, which harms the refractive function of the cornea and can cause a necrosis of the front part of the cornea.

Document EP 0420549 more particularly describes an intracorneal diffractive lens, including a core having a first surface and a second surface opposite the first surface, and at least a first hydrogel layer extending over the first surface of the core and a second hydrogel layer extending over the second surface of the core, the first hydrogel layer including, on the surface thereof facing the core, a plurality of concentric projecting annular areas, each annular area having a continuously varying thickness toward the periphery of the lens.

According to one embodiment described in document EP 0420549, the first and second hydrogel layers are made from hydrogels with a high water content, while the core is made from a hydrogel with a low water content. Making the core from hydrogel with a low water content ensures satisfactory stability of the architecture of the central area of the lens, but considerably harms the nutrient and oxygen permeability in said central area. Furthermore, making the first and second hydrogel layers with a high water content complicates manipulation of the lens.

According to a second embodiment described in document EP 0420549, the first and second hydrogel layers are made from hydrogels with a low water content, while the core is made from a hydrogel with a high water content. Making the core from a hydrogel with a high water content ensures satisfactory nutrient and oxygen permeability in the central area of the lens, but considerably harms the stability of the architecture of said central area.

### BRIEF SUMMARY

The present invention aims to resolve the above-mentioned problems, and therefore aims to provide an intracorneal diffractive lens, adapted to presbytia treatment and designed so as to allow good circulation of the flows of nutrients and oxygen in the thickness of the cornea, when the lens is implanted, while being manipulable and having a stable architecture.

To that end, the invention relates to an intracorneal diffractive lens having phase inversion including a core having a first surface and a second surface opposite the first surface, and at least one first hydrogel layer extending over the first surface of the core and a second hydrogel layer extending over the second surface of the core, the first hydrogel layer including, on the surface thereof turned toward the core, a plurality of concentrically or coaxially projecting annular areas, each annular area having a continuously varying thickness toward the periphery of the lens, characterized in that the first and second layers have a nutrient and oxygen permeability substantially equal to or greater than that of the corneal tissue, and in that at least one of the annular areas of the first layer is in contact with the second hydrogel layer, and in that, for each annular area in contact with the second layer, the distance between the second layer and said annular area varies continuously from a predetermined maximum value to a zero minimum value.

These contact areas between the first and second hydrogel layers allow good circulation of the flows of nutrients and oxygen in the thickness of the cornea, irrespective of the component material of the core.

In fact, when the core is made from a material with a low water content, the circulation of the nutrients and oxygen flows through the lens is ensured at the contact areas between the first and second layers, while the stability of the lens is ensured by the core itself.

When the core is made from a material with a high water content, the stability of the lens is ensured by the contact areas between the first and second layers, which prevent the lens from “collapsing” on itself.

Furthermore, the shape of each annular area in contact with the second layer (due to the continuous variation as far as a zero value of the distance between the latter) ensures satisfactory diffractive behavior of the lens despite the presence of contact areas between the first and second layers in the central area of the lens.

It must be noted that the predetermined maximum value may or may not be identical for each annular area in contact with the second layer.

According to one embodiment of the invention, the first layer is intended to be turned toward the front surface of the cornea and the second layer is intended to be turned toward the rear surface of the cornea. According to another embodiment of the invention, the first layer is intended to be turned toward the rear surface of the cornea and the second layer is intended to be turned toward the front surface of the cornea.

Advantageously, at least one of the annular areas of the first layer is not in contact with the second hydrogel layer. For example, the first layer may comprise a plurality of annular areas in contact with the second layer and a plurality of annular areas situated away from the second layer (i.e. which are not in contact with the second layer), the annular areas in contact with the second layer preferably being regularly distributed on the surface of the lens.

Preferably, the surface of all of the contact areas between the annular areas of the first hydrogel layer and the second hydrogel layer represents less than 20% of the surface of the core, and preferably less than 5% of the surface of the core.

According to one embodiment of the invention, for each annular area in contact with the second layer, the distance between the second layer and each annular area varies continuously from a predetermined maximum value to a zero minimum value toward the periphery of the lens.

Advantageously, the lens according to the invention is an intracorneal diffractive lens with phase inversion, with an analog profile. Such an analog profile contributes, relative to a binary profile, the possibility of choosing the distribution of the light flow between the far focal point and the near focal point so that it is different from the sole equal distribution allowed by the binary profile. Furthermore, the analog profile suffers fewer chromatic aberrations for the extreme wavelengths of the visible spectrum than the binary profile. “Binary profile lens” refers to a lens alternating between optically active annular areas and optically inactive annular areas of similar sizes, and “analog profile lens” refers to a lens having a series of optically active annular areas each corresponding to an average of an optically active area and an optically inactive area of a binary profile.

Advantageously, the first and second layers are made from an interpenetrating polymer network hydrogel including at least a first polymer network and a second polymer network, which are interpenetrating.

This hydrogel, due to its mechanical properties, ensures stability of the architecture of the lens (maintenance of the concentric or coaxial spatial distribution of the annular areas of the first layer), and easy manipulation thereof. Furthermore, such a hydrogel has a significant permeability, in particular to glucose.

Preferably, the first polymer network has a base of polyethylene glycol, and the second polymer network has a base of polyacrylic acid, the polyacrylic acid being polymerized to form the second polymer network in the presence of the first polymer network.

Advantageously, the first and second layers have an optical index substantially identical to that of the cornea.

The core of such a lens may have an optical index higher than that of the first and second layers, or alternatively, lower than that of the first and second layers.

Advantageously, the core is made from a hydrogel, preferably a hydrogel including a polyacrylic acid-based polymer network, or is made up of water. It should be noted that the mechanical properties of the hydrogel making up first and second layers ensure shape stability of the core when the latter is made up of a hydrogel with a high water content or of water.

Preferably, each annular area of the first layer has a thickness increasing continuously toward the periphery of the lens.

Advantageously, each annular area of the first layer has a sinusoidal profile when the core has an optical index higher than that of the first and second layers. Said sinusoidal profile of the annular areas of the first layer makes it possible to obtain a core having diffusion wells ensuring satisfactory permeability of the core despite the fact that the latter has a high optical index. Furthermore, such a sinusoidal profile has a satisfactory optical efficiency.

Preferably, each annular area of the first layer has a parabolic profile when the core has a lower optical index than that of the first and second layers. Such a parabolic profile of each annular area ensures improved optical efficiency.

Preferably, the surface of the second layer turned toward the core is substantially smooth.

Preferably, each contact area between an annular area of the first hydrogel layer and the second hydrogel layer situated in the central portion of the lens has a width substantially smaller than that of said annular area. For example, the or each contact area situated in the central portion of the lens has a width smaller than one quarter of the width of said annular area, or less than one eighth of the width of said annular area. Advantageously, the or each contact area between an annular area and the second hydrogel layer has a width substantially smaller than that of said annular area.

According to one embodiment, the or each contact area between an annular area and the second hydrogel layer is advantageously substantially annular, and preferably annular.

The intracorneal diffractive lens, subject-matter of the invention, can be made as a monofocal lens adapted to correct spherical ametropias, or as a bifocal lens, the latter version being adapted to correct presbytia.

### BRIEF DESCRIPTION OF THE DRAWINGS

In any case, the invention will be well understood using the following description in reference to the appended diagrammatic drawing, illustrating, as non-limiting examples, two embodiments of said lens.

FIG. 1 is a diametric cross-sectional view of an intracorneal diffractive lens according to the present invention, in a first embodiment.

FIG. 2 is a diametric partial cross-sectional view, on an enlarged scale, of an intracorneal diffractive lens according to the present invention, in a second embodiment.

### DETAILED DESCRIPTION

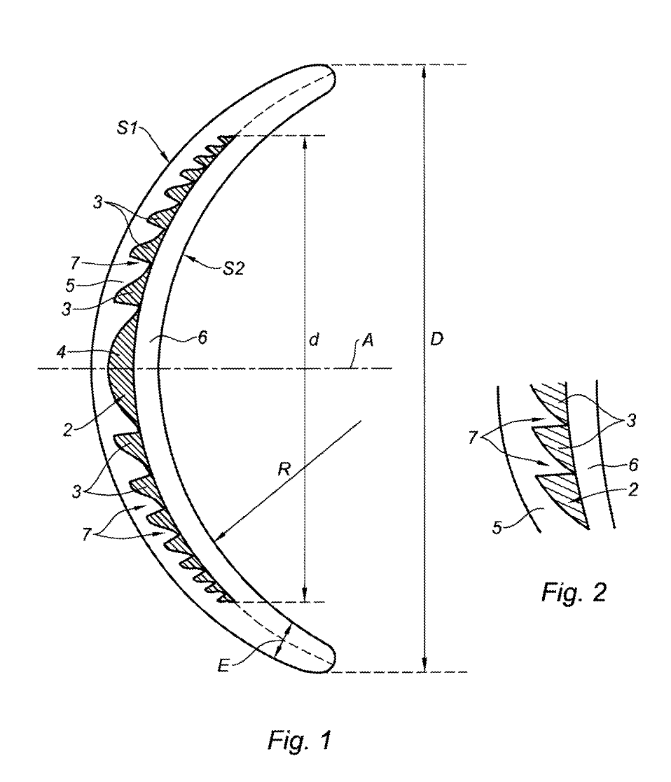

In reference to FIG. 1, an intracorneal diffractive lens whereof the central axis is designated A has an outer diameter D that can be between 5 and 9 mm, and an average curvature defined by a radius R that may be between 7 and 9 mm. This lens has a convex outer surface S1 and a concave inner surface S2, its thickness E measured between the two surfaces S1 and S2 being able to be comprised between 0.02 mm and 0.3 mm.

The useful area of the lens, centered on the axis A, is a circular core 2 whereof the diameter d can be between 3 and 9 mm, depending on the outer diameter D of that lens. This core 2 comprises a series of rings 3, with increasing diameters, all centered on the axis A. The rings 3 have a regularly decreasing width, from the central axis A toward the periphery of the lens, the geometry of the rings 3 being in compliance with the Rayleigh-Wood phase inversion zonal lens principal.

Each ring 3 has a thickness decreasing continuously toward the periphery of the lens. Preferably, the thickness of each ring 3 decreases, toward the periphery of the lens, to a very low value (in the vicinity of several microns) such that the core remains permeable to nutrients in that thinner annular area of each ring 3. Advantageously, the surface of each ring 3 intended to be turned toward the front surface of the cornea of a patient has a sinusoidal profile, and more specifically a profile in the shape of a sinusoidal arc.

In the embodiment illustrated in FIG. 1, the core 2 of the intracorneal lens also includes, in the center thereof, a profiled disk 4 made from the same material as the rings 3, and concentrically or coaxially surrounded by said rings 3. The central disk 4 is comparable to a first ring, with an inner radius equal to zero. As for the rings 3, the central disk 4 has a thickness decreasing continuously toward the periphery of the lens. Preferably, the thickness of the central disk 4 decreases, toward the periphery of the lens, to a very low value (in the vicinity of several microns) such that the core remains permeable to nutrients in the peripheral area of the central disk 4.

The intracorneal lens also includes a first layer 5 and a second layer 6 gripping the core 2. The first layer 5 covers the surface of the core 2 intended to be turned toward the front surface of the cornea of the patient and the second layer 6 covers the surface of the core 2 intended to be turned toward the rear surface of the cornea of the patient, the two layers 5, 6 coming together on the periphery of the lens.

The first and second layers 5, 6 are made from an interpenetrated polymer network hydrogel including a first polymer network with a base of polyethylene glycol and a second polymer network with a base of polyacrylic acid, the polyacrylic acid being polymerized to form the second polymer network in the presence of the first polymer network. The water percentage of the hydrogel is advantageously greater than or equal to 78%.

This hydrogel forms a “cement” that connects all of the rings 3 to one another, thereby stabilizing the structure of the lens. The hydrogel forming the “cement” has a nutrient and oxygen permeability comparable to that of the corneal tissue, and optical index substantially equal to that of the cornea.

The first layer 5 includes, on the surface thereof turned toward the core 2, a plurality of concentric or coaxial protruding annular areas 7 with a thickness increasing continuously toward the periphery of the lens. Each annular area 7 has a profile complementary to that of the corresponding annular area 3 of the core 2.

Advantageously, several annular areas 7 are in contact with the second layer 6. Preferably, the annular areas 7 in contact with the second layer 6 are regularly distributed. For example, every other annular area 7, or every third annular area, is in contact with the second layer 6.

Preferably, each contact area between an annular area 7 and the second hydrogel layer 6 has a width smaller than one quarter of the width of said annular area 7, or smaller than one eighth of the width of said annular area 7.

Preferably, the surface of the second layer 6 turned toward the core 2 is substantially smooth.

The core 2, i.e. the rings 3 and the central disk 5, is made from a material having a different optical index from that of the cornea. In the embodiment of FIG. 1, this may also involve a hydrogel, but whereof the optical index is higher than that of the hydrogel making up the first and second layers and whereof the water percentage is less than 78%, and preferably between 50% and 70%. The hydrogel making up the core 2 can preferably be a hydrogel including a polymer network with a base of polyacrylic acid.

The rings 3, of which there may be between five and thirty (the drawing showing, in a simplified matter, a very small number of rings), have a lower permeability than that of the cornea, and cause, with the central disk 5, the diffraction necessary for the desired vision correction.

The outer S1 and inner S2 surfaces can be parallel, therefore without any effect on the correction done, or on the contrary may be non-parallel and configured so as to participate in the visual correction, through an additional refractive effect.

Such an intracorneal diffractive lens, combining two materials, can be made using molding or overmolding techniques. In particular, it may be manufactured for a dual injection method.

Advantageously, the method for manufacturing the lens shown in FIG. 1 includes the following steps:
introducing a polyethylene glycol-based aqueous solution into a first mold transparent to UV rays,
plugging the first mold using a stopper having, on the surface thereof intended to be pressed against the upper surface of the aqueous solution, a profile corresponding to that of the surface of the core intended to be turned toward the front surface of the cornea of the patient,
exposing the first mold to UV rays in order to polymerize the polyethylene glycol so as to obtain a first solid layer made of a hydrogel including a polyethylene glycol-based polymer network,
introducing a polyethylene glycol-based aqueous solution into a second mold transparent to UV rays,
plugging the second mold using a stopper having, on the surface thereof intended to be pressed against the upper surface of the aqueous solution, a profile corresponding to that of the surface of the core intended to be turned toward the rear surface of the cornea of the patient,
exposing the second mold to the UV rays in order to polymerize the polyethylene glycol so as to obtain a second solid layer formed from a hydrogel including a polyethylene glycol-based polymer network,
superimposing the first and second layers in a third mold and injecting, into the third mold, a polyacrylic acid-based aqueous solution,
exposing the third mold to the UV rays in order to polymerize the polyacrylic acid so as to obtain, on the one hand, the first and second layers 5, 6 formed from an interpenetrating polymer network hydrogel including a first polymer network with a base of polyethylene glycol and a second polymer network with a base of polyacrylic acid, and on the other hand, the core 2 formed from a hydrogel including a polymer network with a base of polyacrylic acid.

Such a manufacturing method ensures perfect cohesion between the first and second layers 5, 6 and the core 2 as well as perfect adhesion thereof, which further improves the stability of the architecture of the lens.

FIG. 2, in which the elements corresponding to those previously described are designated using the same references, shows an alternative of said intracorneal diffractive lens. In this alternative, the surface of each ring 3 intended to be turned toward the front surface of the cornea of a patient has a convex and parabolic profile, and more specifically a convex profile in the shape of a parabola arc. Furthermore, according to this alternative, the core 2 has a lower optical index than that of the first and second layers 5, 6. In that case, the first and second layers are made from a hydrogel whereof the water content is close to 78%, while the core 2 is made from a hydrogel whereof the water content is higher than that of the hydrogel making up the first and second layers, and typically greater than 85%, or made up of water.

The invention is of course not limited to the sole embodiments of this intracorneal diffractive lens described above as examples, but on the contrary encompasses all alternative embodiments within the scope of the claims.

1. An intracorneal diffractive lens having phase inversion including a core having a first surface and a second surface opposite the first surface, and at least one first hydrogel layer extending over the first surface of the core and a second hydrogel layer extending over the second surface of the core, the first hydrogel layer including, on the surface thereof turned toward the core, a plurality of concentrically or coaxially projecting annular areas, each annular area having a continuously varying thickness toward a periphery of the lens, wherein the first and second layers have a nutrient and oxygen permeability substantially equal to or greater than that of the corneal tissue, and wherein at least one of the annular areas of the first layer is in contact with the second hydrogel layer, and wherein, for each annular area in contact with the second layer, the distance between the second layer and said annular area varies continuously from a predetermined maximum value to a zero minimum value.

2. The intracorneal lens according to claim 1, wherein the surface of all of the contact areas between the annular areas of the first hydrogel layer and the second hydrogel layer represents less than 20% of the surface of the core.

3. The intracorneal lens according to claim 1, wherein the first and second layers are made from an interpenetrating polymer network hydrogel including at least a first polymer network and a second polymer network, which are interpenetrating.

4. The intracorneal lens according to claim 3, wherein the first polymer network has a base of polyethylene glycol, and the second polymer network has a base of polyacrylic acid, the polyacrylic acid being polymerized to form the second polymer network in the presence of the first polymer network.

5. The intracorneal lens according to claim 1, wherein the first and second layers have an optical index substantially identical to that of the cornea.

6. The intracorneal lens according to claim 5, wherein the core has an optical index higher than that of the first and second layers.

7. The intracorneal lens according to claim 5, wherein the core has an optical index lower than that of the first and second layers.

8. The intracorneal lens according to claim 1, wherein the core is made from a hydrogel including a polyacrylic acid-based polymer network, or is made up of water.

9. The intracorneal lens according to claim 1, wherein each annular area of the first layer has a thickness increasing continuously toward the periphery of the lens.

10. The intracorneal lens according to claim 1, wherein the first layer may comprise a plurality of annular areas in contact with the second layer and a plurality of annular areas situated away from the second layer, the annular areas in contact with the second layer preferably being regularly distributed on the surface of the lens.

11. The intracorneal lens according to claim 1, wherein each annular area of the first layer has a sinusoidal or parabolic profile.

12. The intracorneal lens according to claim 1, wherein the surface of the second layer turned toward the core is substantially smooth.

13. The intracorneal lens according to claim 1, wherein the or each contact area between an annular area of the first hydrogel layer and the second hydrogel layer is advantageously substantially annular.
